(19) United States
(12) Patent Application Publication
(10) Pub. No.: US 2022/0099673 A1
(43) Pub. Date: Mar. 31, 2022

(54) Biosensor, bio-sensing system comprising the same and method for preparing the same

(71) Applicant: Academia Sinica, Taipei City (TW)

(72) Inventors: Chia-Fu CHOU, Taipei City (TW); Stalin KARUPPIAH, Taipei City (TW)

(21) Appl. No.: 17/485,815

(22) Filed: Sep. 27, 2021

Related U.S. Application Data
(60) Provisional application No. 63/084,022, filed on Sep. 28, 2020.

(51) Int. Cl. G01N 33/569; G01N 27/327

(52) U.S. Cl. CPC G01N 33/56983; G01N 27/327; G01N 2333/165; G01N 2333/42; G01N 2800/26

(57) ABSTRACT

A biosensor is provided, which comprises: a substrate; a working electrode disposed on the substrate and comprising a graphene layer; a counter electrode disposed on the substrate and adjacent to the working electrode; a reference electrode disposed on the substrate and adjacent to the working electrode; and a bio-recognition layer disposed on the working electrode. In addition, a bio-sensing system comprising the aforesaid biosensor and a method for preparing the aforesaid biosensor are also provided.

### CROSS REFERENCE TO RELATED APPLICATION

This application claims the benefit of filing date of U.S. Provisional Application Ser. No. 63/084,022, filed Sep. 28, 2020 under 35 USC § 119(e)(1).

### BACKGROUND

### 1. Field

The present disclosure relates to a biosensor, a bio-sensing system comprising the same and a method for preparing the same.

### 2. Description of Related Art

A biosensor is an analytical device that can convert the concentration or amount of any given biological analytes into measurable signals such as optical, electrical, electrochemical etc., or other signals for detection. There are many potential applications of biosensors of various types, such as environmental assessment and monitoring (e.g. the detection of river water contaminants) and detection of pathogens.

For example, water-borne pathogens are mostly generated due to poor sanitation or industrial effluents, sewage sludge leads to a significant increase in the mortality rate. Thus, a simple, user-friendly, and rapid on-site detection tool of the pathogens, i.e., biosensor, has to be developed to prevent this.

In addition, the recently emerged coronavirus disease, COVID-19, caused by severe acute respiratory syndrome coronavirus 2 (SARS-CoV2), leads to distress in breathing and pneumonia. The extreme contagiousness and number infections due to this disease has prompted the World Health Organization to classify this outbreak as a pandemic. Thus, a biosensor with remarkable sensitivity, specificity and rapid testing ability has to be developed for rapid detection and quantification of SARS-CoV2 to delay the spread of this disease.

Therefore, it is desirable to provide a novel biosensor which can effectively detect the target biomolecules in the analytes.

### SUMMARY

One object of the present disclosure is to provide a biosensor and, in particular, a portable biosensor with high sensitivity.

The biosensor of the present disclosure comprises: a substrate; a working electrode disposed on the substrate and comprising a graphene layer; a counter electrode disposed on the substrate and adjacent to the working electrode; and a bio-recognition layer disposed on the working electrode.

The present disclosure further provides a method for preparing the aforesaid biosensor, which comprises the following steps: providing a substrate; forming a working electrode and a counter electrode on the substrate, wherein the counter electrode is adjacent to the working electrode, and the working electrode comprises a graphene layer; and forming a bio-recognition layer on the working electrode.

In the method of the present disclosure, the method may further comprise a step of forming a modification layer on the graphene layer before the step of forming the bio-recognition layer on the working electrode, wherein the modification layer comprises graphene oxide. Thus, in the biosensor of the present disclosure, the working electrode may further comprise a modification layer disposed on the graphene layer and comprising graphene oxide.

In the biosensor of the present disclosure, the modification layer comprising graphene oxide is formed on the surface of the graphene layer of the working electrode, and bio-recognition elements in the bio-recognition layer can bind to the modification layer through covalent attachments. By the covalent attachment between the bio-recognition elements and the graphene oxide of the modification layer, the biosensor of the present disclosure has higher sensitivity than the conventional biosensor that the bio-recognition elements bind to the working electrode through physisorption. Thus, even though the amount of the biomolecules in the sample to be detected is low, the ultrasensitive biosensor of the present disclosure still can effectively detect the biomolecules in the sample.

In one embodiment of the biosensor of the present disclosure, the working electrode may comprise the graphene layer and a modification layer disposed on the graphene layer. More specifically, the working electrode may be a graphene oxide modified graphene electrode. The working electrode can be formed on the substrate by any coating process known in the art, for example, a screen-printing process. In one embodiment of the present disclosure, the working electrode can be formed by applying a graphene ink on the substrate via the screen-printing process, followed by applying a graphene oxide ink on the graphene layer, but the present disclosure is not limited thereto.

In another embodiment of the biosensor of the present disclosure, the working electrode may comprise the graphene layer but does not comprise a modification layer (for example, the graphene oxide layer). More specifically, the working electrode may be a graphene electrode. Similarly, the working electrode can be formed on the substrate by any coating process known in the art, for example, a screen-printing process. In one embodiment of the present disclosure, the working electrode can be formed by applying a graphene ink on the substrate via the screen-printing process, but the present disclosure is not limited thereto.

In the biosensor of the present disclosure, the counter electrode may comprise a material which should not corrode in the medium/electrolyte used for the detection (for example, W, Au, Pt, Ti, or an alloy thereof) or a conductive metal oxide (for example, ITO, IZO, ITZO, IGZO, or AZO).

In the biosensor of the present disclosure, the counter electrode may comprise graphene. More specifically, the counter electrode may be a graphene electrode. The counter electrode can be formed on the substrate by any coating process known in the art, for example, a screen-printing process. In one embodiment of the present disclosure, the counter electrode can be formed by applying a graphene ink on the substrate via the screen-printing process, but the present disclosure is not limited thereto.

In the method for preparing the biosensor of the present disclosure, the graphene layer or the graphene electrode can be prepared by the screen-printing process, and thus the biosensor of the present disclosure can be prepared easily, simply or quickly, or can be mass-produced.

In the biosensor of the present disclosure, the modification layer of the working electrode may comprise graphene oxide. More specifically, the modification layer is a graphene oxide layer. Most specifically, when the working electrode comprises the graphene layer and the modification layer on the graphene layer, the modification layer is a graphene oxide layer enriched with carboxylic acid (—COOH) group. Herein, the graphene oxide layer may comprise mono or few molecular layers of the graphene oxide. In addition, the modification layer can be formed on the working electrode by any process known in the art, for example, a drop casting process, but the present disclosure is not limited thereto.

In the biosensor of the present disclosure, when the working electrode comprises the graphene layer but does not comprise the modification layer (i.e. the graphene oxide layer), the graphene layer may be modified to be enriched with carboxylic acid (—COOH) group.

The biosensor of the present disclosure may further comprise a reference electrode disposed on the substrate and electrically isolated from the working electrode. Herein, the reference electrode may be an Ag electrode or an Ag—AgCl composite electrode. In one embodiment of the present disclosure, the reference electrode may be prepared by depositing Ag on the substrate to form the Ag electrode. In another embodiment of the present disclosure, the reference electrode may be prepared by depositing Ag on the substrate, followed by converting partial Ag into AgCl to form the Ag—AgCl composite electrode.

In the biosensor of the present disclosure, the substrate may include paper, glass, silicon wafer, sapphire, polycarbonate (PC), polyimide (PI), polypropylene (PP), polyethylene terephthalate (PET), poly(methyl methacrylate) (PMMA), any suitable polymer or a combination thereof. In one embodiment of the present disclosure, the substrate is a paper-based substrate, in particular, a hydrophobic paper-based substrate. When the paper-based substrate is used, the prepared biosensor is cost-effective or eco-friendly. In another embodiment of the present disclosure, the substrate is a PET-based substrate.

In the biosensor of the present disclosure, a bio-recognition layer is disposed on the working electrode, wherein the bio-recognition layer comprises bio-recognition elements for recognizing the biomolecules in the sample to be detected. Herein, the working electrode may be connected to the bio-recognition layer through a linker. In one embodiment of the present disclosure, the linker is —CONH—. Herein, one end of —CONH— is covalently bonded to the working electrode, and the other end of —CONH— is covalently bonded to the bio-recognition elements in the bio-recognition layer.

In the biosensor of the present disclosure, the bio-recognition layer may comprise any bio-recognition elements capable of recognizing the biomolecules in the sample to be detected. Examples of the bio-recognition elements may include, but are not limited to, tissue, microorganisms, organelles, cell receptors, enzymes, antibodies, nucleic acids, etc. In one embodiment of the present disclosure, the bio-recognition layer may comprise lectin, which may be Concanavalin A (ConA) based lectin. In another embodiment of the present disclosure, the bio-recognition layer may comprise an antibody, which may be an anti-severe acute respiratory syndrome coronavirus-2 (anti-SARS-CoV2) antibody such as anti-SARS-CoV2 spike antibody. However, the present disclosure is not limited thereto, and the bio-recognition elements may be selected according to the biomolecules to be detected.

In addition to the biosensor and the method for preparing the same, the present disclosure further provides a bio-sensing system comprising the aforesaid biosensor. The bio-sensing system of the present disclosure comprises: the aforesaid biosensor; and a detection device receiving signals from the biosensor.

Herein, the used detection device is not particularly limited. In one embodiment of the present disclosure, the detection device may be an impedance analyzer. In another embodiment of the present disclosure, the detection device may be a voltammetry, such as a differential pulse voltammetry (DPV) or a square-wave voltammetry (SWV). However, the present disclosure is not limited thereto.

The present disclosure further provides a detecting method, which comprises the steps of: providing the aforesaid biosensor; applying a sample to be detected onto the biosensor; and reading out signals from the biosensor by a detection device. Herein, when significant signals are observed, it means that the sample comprises the biomolecules to be detected. In addition, when the observed signals are compared with a standard, it is possible to quantify the biomolecules to be detected in the sample.

Other novel features of the disclosure will become more apparent from the following detailed description when taken in conjunction with the accompanying drawings.

### BRIEF DESCRIPTION OF THE DRAWINGS

FIG. 1A is a schematic diagram of a biosensor according to one example of the present disclosure.

FIG. 1B is a cross-sectional view of a biosensor according to one example of the present disclosure.

FIG. 2 shows an analytical performance of a biosensor prepared in Example 1 for the sensing of single sized bacteria.

FIG. 3 shows an analytical performance of a biosensor prepared in Example 1 for the sensing of difference size bacteria.

FIG. 4 shows an analytical performance of a biosensor prepared in Example 1 for the sensing of grain-positive bacteria.

FIG. 5 shows a plot of logarithmic concentration of the bacterial cell vs. charge transfer resistance for single sized bacteria.

FIG. 6 shows a plot of logarithmic concentration of the bacterial cell vs. charge transfer resistance for difference size bacteria.

FIG. 7 shows a plot of OD measurement vs. concentration of the bacterial cell.

FIG. 8 shows an analytical performance of a biosensor prepared in Example 2 using differential pulse voltammetry (DPV).

FIG. 9 shows an analytical performance of a biosensor prepared in Example 2 using square wave voltammetry (SWV).

FIG. 10 shows an analytical performance of a comparative sensor using differential pulse voltammetry (DPV).

FIG. 11 shows the current vs. anti-SARS-CoV2 concentration curves of a biosensor prepared in Example 2 obtained by DPV based method.

FIG. 12 shows the current vs. anti-SARS-CoV2 concentration curves of a biosensor prepared in Example 2 obtained by SWV based method.

FIG. 13 shows an analytical performance of a biosensor prepared in Example 2 using differential pulse voltammetry (DPV).

FIG. 14 shows another analytical performance of a biosensor prepared in Example 2 using differential pulse voltammetry (DPV).

### DETAILED DESCRIPTION OF EMBODIMENT

Different embodiments of the present disclosure are provided in the following description. These embodiments are meant to explain the technical content of the present disclosure, but not meant to limit the scope of the present disclosure. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not means that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially means the existent of another element described by a smaller ordinal number.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially means that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

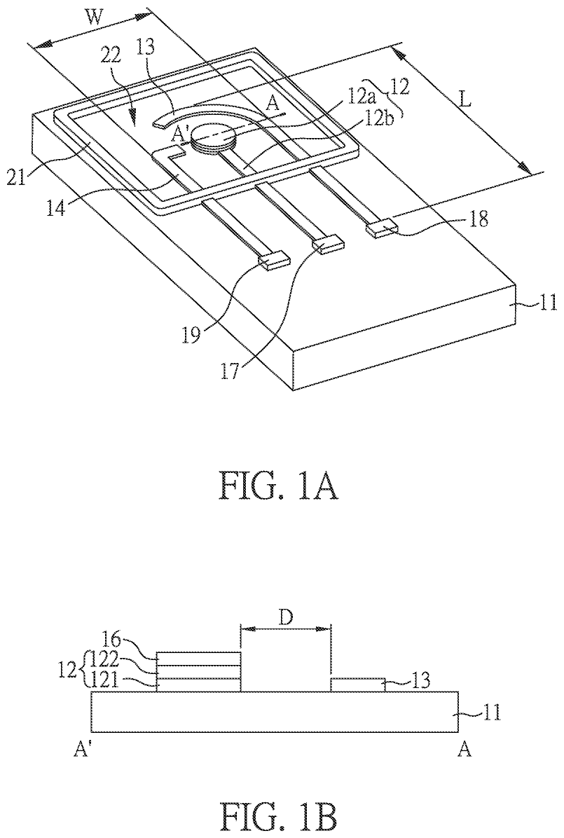

FIG. 1A is a schematic diagram of a biosensor according to one example of the present disclosure, and FIG. 1B is a cross-sectional view of the biosensor along the line A-A′ indicated in FIG. 1A.

The biosensor of one example of the present disclosure may be prepared by the following steps. First, a substrate 11 is provided. Then, a working electrode 12 and a counter electrode 13 are formed on the substrate 11, wherein the counter electrode 13 is adjacent to the working electrode 12, and the working electrode 12 comprises a graphene layer 121. Herein, the graphene layer 121 of the working electrode 12 or the counter electrode 13 may be prepared by a screen-printing process with a graphene ink.

After forming the graphene layer 121 of the working electrode 12 and the counter electrode 13, a modification layer 122 is formed on the graphene layer 121 of the working electrode 12, wherein the modification layer 122 may be prepared by a drop casting process with a solution containing graphene oxide. After forming a bio-recognition layer 16 on the modification layer 122, the biosensor of the present example is obtained.

Herein, one biosensor is fabricated at a time, but the present disclosure is not limited thereto. In another example of the present disclosure, plural biosensors may be fabricated at the same time.

For example, a mother substrate is provided, and plural graphene layers 121 of plural working electrodes 12 and plural counter electrode 13 are formed on the mother substrate. Then, the modification layer 122 is formed on each of the plural graphene layers 121, followed by forming the bio-recognition layer 16 on each of the modification layer 122. After cutting the mother substrate into plural substrates 11, plural independent biosensors as shown in FIG. 1A can be obtained. Thus, it is possible to mass-produce biosensors.

After the aforesaid process, the biosensor can be obtained, which comprises: a substrate 11; a working electrode 12 disposed on the substrate 11 and comprising a graphene layer 121; a counter electrode 13 disposed on the substrate 11 and adjacent to the working electrode 12; a modification layer 122 disposed on the graphene layer 121 of the working electrode 12 and comprising graphene oxide; and a bio-recognition layer 16 disposed on the modification layer 122. Herein, the modification layer 122 may be connected to the bio-recognition layer 16 through a linker, such as —CONH—.

In addition, the biosensor further comprises: a reference electrode 14 disposed on the substrate 11 and electrically isolated from the working electrode 12.

Herein, the working electrode 12 may be a graphene oxide modified graphene electrode, the counter electrode 13 may be a graphene electrode, and the reference electrode 14 may be an Ag electrode or an Ag—AgCl composite electrode.

Furthermore, the working electrode 12 comprises: a working region 12a adjacent to the counter electrode 13; and a conductor region 12b connecting to the working region 12a. Herein, the modification layer 122 is disposed on the working region 12a, and the bio-recognition layer 16 is further disposed on the modification layer 122 on the working region 12a.

In addition, the biosensor may further comprise: a first pad 17 electrically connected to the working electrode 12; a second pad 18 electrically connected to the counter electrode 13; and a third pad 19 electrically connected to the reference electrode 14. Herein, the first pad 17, the second pad 18 and the third pad 19 are used to connect to a detection device (not shown in the figure) when a sample is to be detected, and the signals of the working electrode 12 and the counter electrode 13 can transmit to the detection device through the first pad 17 and the second pad 18. The material of the first pad 17, the second pad 18 and the third pad 19 is not particularly limited, and may be Cu, Al, Mo, W, Au, Cr, Ni, Pt, Ti, or an alloy thereof.

In addition, the biosensor may further comprise: a dam 21 for forming a chamber 22, wherein the working electrode 12, the counter electrode 13 and the reference electrode 14 locate in the chamber 22, and a sample to be detected can be dropped into the chamber 22.

### Example 1

### Synthesis of Graphene Oxide (GO)

0.5 g of graphite was added to 23 mL H₂SO₄ (98%) with stirring under 0-4° C., then 0.5 g of NaNO₃ was added rapidly followed by slow addition of finely divided KMnO₄ (1.5 g) into the reaction mixture under stirring. The reaction was continued until the dark green coloration was observed. Then the solution was transferred to an oil bath, and maintained at 37° C. under vigorous stirring for 2 h. Thereafter, 50 mL of deionized water was added dropwise through a funnel while stirring the mixture, and then the mixture was refluxed and maintained at 95° C. for about 45 minutes. The refluxing step is introduced in order to have a crowd of —COOH groups on the 2D surface of GO, and the presence of —COOH groups helps to stabilize the GO dispersions by lowering the pKₐ value. The above modification in the synthesis indirectly enables the ease of activation by —COOH to —CONH bond formation through EDC/NHS coupling which in turn highly facilitates the specificity of the lectin immobilization onto the 2D GO surface and avoids the non-specific attachment of lectin. Subsequently, the reaction mixture was poured into 125 mL deionized water (DI) followed by the addition of 2.5 ml of H₂O₂ (30%). After adding H₂O₂, the sudden change in color from dark brown to yellow confirmed the termination of oxidation process. To remove the metal ions, the obtained GO suspension was washed with aqueous solution of HCl (1:9 (v/v)) through centrifugation at 6000 rpm for 10 minutes. The GO suspension was centrifuged several times in DI water until it reaches the neutral pH. The neutral GO suspension was sonicated for 2 h and freeze-dried to get highly exfoliated sheets. The resultant GO appeared as a yellow coloured fluffy substance.

### Preparation of Biosensor

In the present example, the substrate 11 shown in FIG. 1A was a paper-based substrate, which was hydrophobic, hot pressed paper (Cheng Tien Intl. Corp., Taiwan). The reference electrode 14 shown in FIG. 1A was an Ag/AgCl reference electrode, which was prepared by screen-printing using Ag/AgCl paste.

Conductive graphene (G) screen-printing ink was purchased from Haydale Co. Ltd. Hydrophobic. Screen-printing was done by using ATMA AT-25PA flat screen-printer by ATMA Champ Ent. Corp., Taiwan. The working electrode of the biosensor of the present example was prepared as follows.

Graphene ink was screen-printed on the paper to have a graphene screen-printing electrode (GSPE). Two layers of printing have been done to obtain the electrode with low resistance. The measured resistance for single and double layers is found to be 50 Ωcm⁻¹ and 30 Ωcm⁻¹, respectively. Each layer was cured at room temperature for about 10 minutes. After the aforesaid process, the graphene layer 121 of the working electrode 12 and the counter electrode 13 shown in FIG. 1A were obtained, which are respectively a graphene screen-printing electrode (GSPE).

Then, the modification layer 122 shown in FIG. 1A was fabricated by drop casting 20 μL of 1 mg/mL suspension of GO in DI water onto the GSPE, then allowed to dry at room temperature, washed with DI water, and dried in N₂. The HRTEM images of GO (not shown in the figure) show translucence, wrinkles and folds in the GO sheets. This indicates that the synthesized GO is two dimensional in nature and comprises of mono or few molecular layers of the GO.

A micro-well reaction chamber of total volume of 50 μL (i.e. the chamber 22 shown in FIG. 1A) was created by 3M tape (i.e. the dam 21 shown in FIG. 1A) through punching technique.

The bio-recognition layer 16 shown in FIG. 1A was fabricated as follows. ConA was immobilized on the graphene oxide layer (i.e. the modification layer 122 shown in FIG. 1A) on the graphene layer (i.e. the graphene layer 121 of the working electrode 12 shown in FIG. 1A) by the activation of carboxyl groups present in the GO using EDC/NHS chemistry. Briefly, EDC (0.5 M) and NHS (0.1 M) solution was freshly prepared in MES buffer and the modification layer 122 was incubated in 1:1 (v/v) ratio of EDC/NHS by dropping 15 μL solution of EDC/NHS. Then, the electrode was incubated for about 30 minutes at room temperature in 10 μL of lectin (ConA) prepared in sodium acetate buffer (0.1 M, 0.2 mg/mL) of pH 4.5. Afterwards, the non-specifically adsorbed sites were blocked by immersing the electrode in ethanolamine solution (0.1 M) for 20 minutes. The electrode was washed with DI water after each step and dried in N₂ gas.

After the aforesaid process, the biosensor of the present example is obtained. As shown in FIG. 1A and FIG. 1B, the biosensor of the present example has a length L of 22 mm, a width of 12 mm, and an inter-electrode distance D (between the working electrode 12 and the counter electrode 13) of 2 mm. The working region 12a of the working electrode 12 is circular in geometry with the electroactive surface area of 0.16 cm². However, the present disclosure is not limited thereto, and the aforesaid values can be adjusted according to the need.

### Analytical Performance of Biosensor

Synthetic waste water was prepared with a composition of 2 mM (NH₄)₂SO₄, 0.2 mM MgSO₄.7H₂O, 0.03 mM MnSO₄.H₂O, 1.5 mM NaHCO₃, 0.01 mM FeCl₃.6H₂O, 0.03 mM MgCl₂.2H₂O and 100 mM CH₃COOK. Bacterial cultures of W3110 K-12, in synthetic waste water, were incubated overnight at 37° C. The cultures, after overnight growth, had been filtered through 100 μm filter paper to remove the largely suspended solids and other organisms, while allowing bacteria to pass through. The colonies were counted by the microbial plate count method and found to be approximately 10⁹ CFU mL⁻¹ in the overnight culture, which was used as stock. Samples with different bacterial concentrations were obtained by serial dilution of this stock solution in sterile PBS solution (PBS, 0.1 M, pH 7.4).

Electrochemical impedance spectroscopy (EIS) measurements were recorded at a potential of 0.2 V in the frequency range of 0.1 Hz to 1 MHz with amplitude of 0.01 V. All the electrochemical experiments were conducted in 0.1 M sterile phosphate buffer (PBS) in presence of the redox probe, 5 mM [Fe(CN)₆]^{3-/4-}.

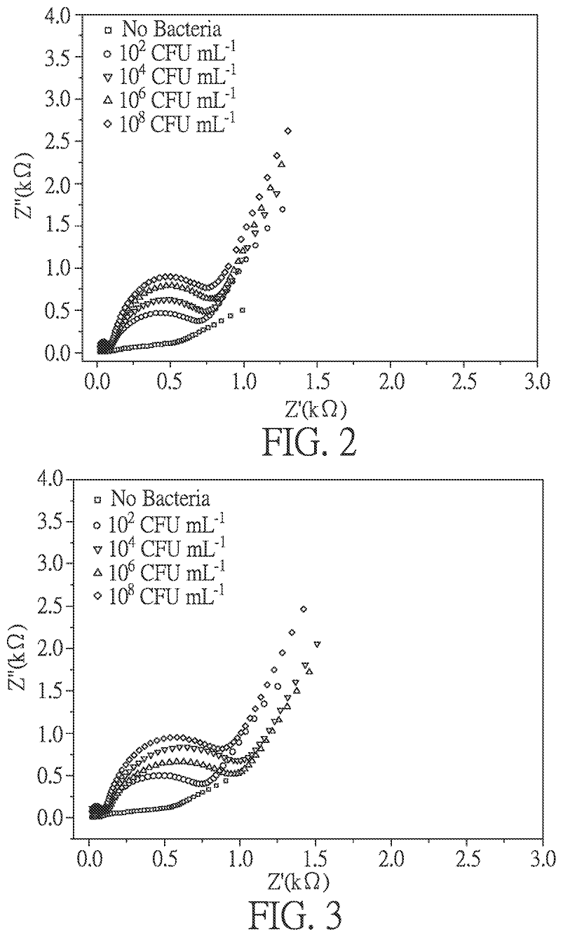

FIG. 2 to FIG. 4 show analytical performances of a biosensor prepared in Example 1 for the sensing of single sized bacteria, difference size bacteria and grain-positive bacteria respectively.

The EIS response shown in FIG. 2 showed an exponential increase in the interfacial electron transfer resistance with the concentration of the bacteria cells. This trend was observed by the increase in charge transfer resistance (R_{ct}) presented in FIG. 2. The high sensitivity of the biosensor to the bacterial cells may be due to extensive covalent immobilization of ConA to the large area of graphene oxide surface where plenty of —COOH groups are present to facilitate the binding of the ConA. The self-assembly of lectin ConA through physisorption lacks the specificity in detection, but the covalent attachment increases the quality of the biosensor by the increase in lifetime and sensitivity.

As shown in FIG. 3 and FIG. 4, the EIS measurements were conducted for grain-positive bacteria and bacteria of different size distribution to test the selectivity of the prepared biosensor. The results shown in FIG. 3 and FIG. 4 were corroborated that there was no perturbation in the selectivity and sensitivity of the biosensor as it only relies on the total concentration of bacteria, whose surface is highly specific to the ConA binding. It was attributed by the observation that the grain-positive bacteria differ from the grain-negative only by a thin layer of peptidoglycan, which also contains the ConA binding sugars on its surface, thus resulting in no significant change in R_{ct} values. In the case of bacteria size influence, the biosensor designed in such a way that the surface of the ConA bound electrode accommodates the specific concentration (CFU mL⁻¹) of bacteria irrespective of their size.

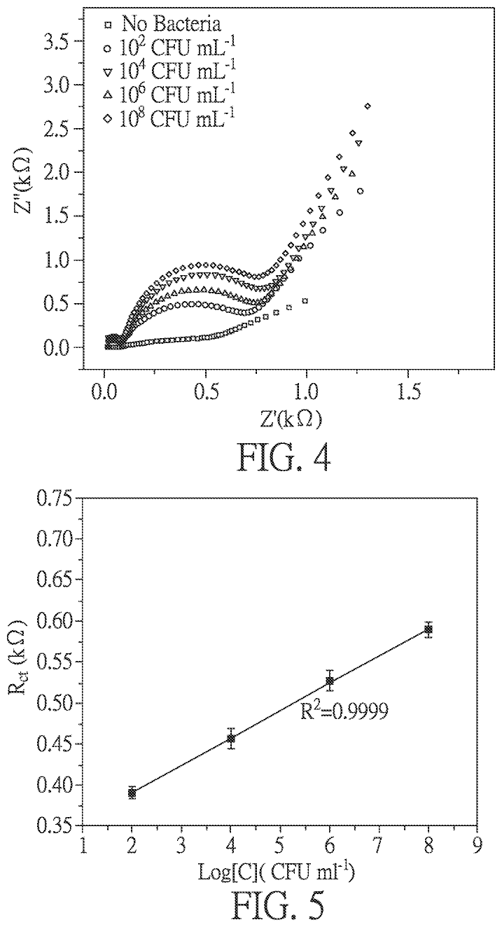

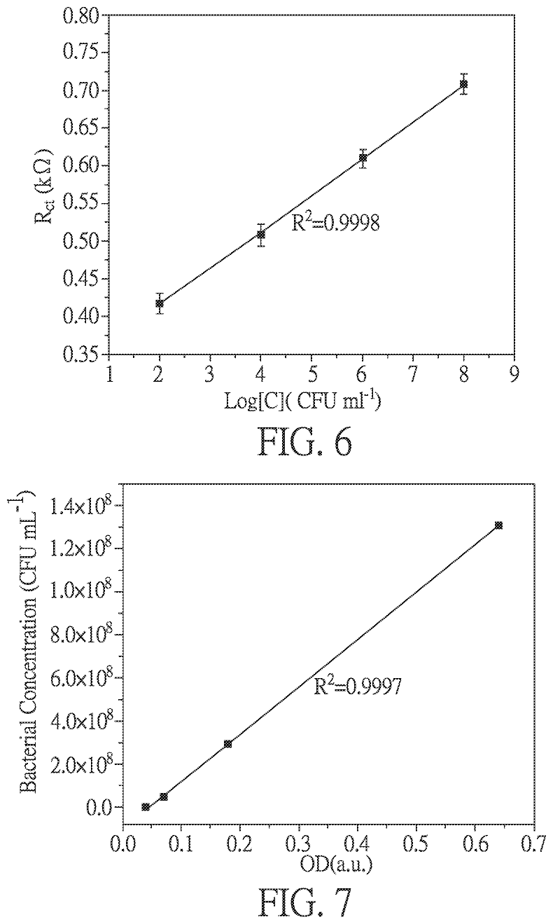

A plot of logarithmic concentration of the bacterial cell vs. charge transfer resistance was plotted to calculate the limit of detection (LOD) and standardize the biosensor. The results are shown in FIG. 5 and FIG. 6.

FIG. 5 and FIG. 6 show plots of logarithmic concentration of the bacterial cell vs. charge transfer resistance for single sized bacteria and difference size bacteria respectively.

The results shown FIG. 5 and FIG. 6 indicate a good linear correlation with linear range (LR) of 10²-10⁸ CFU mL⁻¹, and the impedance of the biosensor was saturated above the linear range mentioned. The limit of detection (LOD) of the fabricated biosensor was calculated and found to be 10 CFU mL⁻¹ and the limit of quantitation (LOQ) is 23.25 CFU mL⁻¹.

Meanwhile, a plate count method to quantify the bacterial colonies was conducted simultaneously along with the impedimetric detection. The obtained results (as shown in FIG. 7) of the plate count experiments on bacteria were plotted against OD, which proves that the results of the proposed electrochemical impedimetric sensing method of the present disclosure have good agreement with the bacterial concentration (CFU mL⁻¹) measured by plate count method.

From the results shown above, the fabricated biosensor of the present example is found to have the low LOD with the wide LR. This is due to the large surface area of GO with plenty of —COOH groups which bind a large number ConA molecules on the active sites of the working electrode, and in turn facilitate the bacteria cells to the ConA molecules effectively.

The results presented here established the robustness, efficiency, versatility, and sensitivity of the developed paper-based bacterial biosensor. The fabrication method of using screen-printing is simple to operate, cost-effective and avoids the usage of toxic photoresists which is employed in lithographic techniques. Moreover, the hydrophobic paper, used as a substrate, is inexpensive and eco-friendly. Therefore, the prepared biosensor having extremely low LOD presents great potential in applications of bacterial biosensing.

### Example 2

The biosensor of the present example is similar to that shown in Example 1, except for the following differences.

In the present example, the substrate 11 shown in FIG. 1A is a PET substrate, and the bio-recognition elements in the bio-recognition layer 16 shown in FIG. 1A are anti-SARS-CoV2 spike antibodies purchased from GenTex, Taiwan (R.O.C).

The Ag/AgCl electrode (i.e. the reference electrode 14 shown in FIG. 1A), the graphene layers (i.e. the graphene layer 121 of the working electrode 12 and the counter electrode 13) and the graphene oxide layer (i.e. the modification layer 122 of the working electrode 12) of the biosensor of the present example were prepared by the similar process illustrated in Example 1. The bio-recognition layer 16 shown in FIG. 1A was fabricated as follows.

Herein, anti-SARS-CoV2 was immobilized on the graphene oxide layer (i.e. the modification layer 122 shown in FIG. 1A) on the graphene electrode (i.e. the graphene layer 121 of the working electrode 12 shown in FIG. 1A) by the activation of carboxyl groups present in the GO using EDC/NHS chemistry. Briefly, an EDC (0.5 M) and NHS (0.1 M) solution was freshly prepared in 1×PBS buffer. Then, a 5 μL EDC/NHS solution in a 1:1 (v/v) ratio was dropping onto the graphene oxide layer followed by incubation. Then, the electrode was incubated for approximately 30 minutes at room temperature in 5 μL of anti-SARS-CoV2 (20 μG mL⁻¹) prepared in 1×PBS buffer at pH 7.2. Afterwards, the nonspecifically adsorbed sites were blocked by immersing the electrode into BSA solution in 1×PBS for 15 minutes. The electrode was washed with DI water after each step and dried in N₂ gas.

### Analytical Performance of Biosensor

Herein, the fabricated biosensor against the SARS-CoV2 spike protein (purchased from GenTex, Taiwan, R.O.C., and Sino Biologicals, Taiwan, R.O.C.) was tested using square wave voltammetry (SWV) and differential pulse voltammetry (DPV) techniques.

The pilot experiments involved the detection and quantification of the spike protein in PBS to determine the limit of detection (LOD) of the fabricated biosensor. The analytical performance of the fabricated biosensor was tested in presence of the common interfering proteins and enzyme(s) present in nasal cavities.

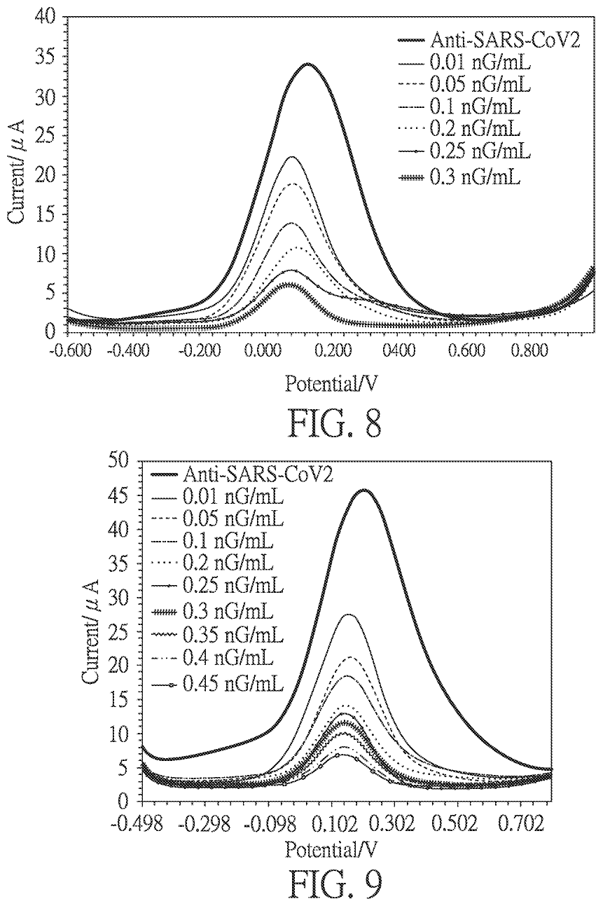

FIG. 8 and FIG. 9 respectively show the analytical performances of the biosensor using DPV and SWV, wherein Anti-SARS-CoV2 refers to the antibody of SARS-CoV2 as a negative control. The result show that the current response falls steeply with increasing SARS-CoV2 spike protein concentration, suggesting that the more the protein binds the less the electron transfer, which, in turn, leads to a fall in current in DPV and SWV.

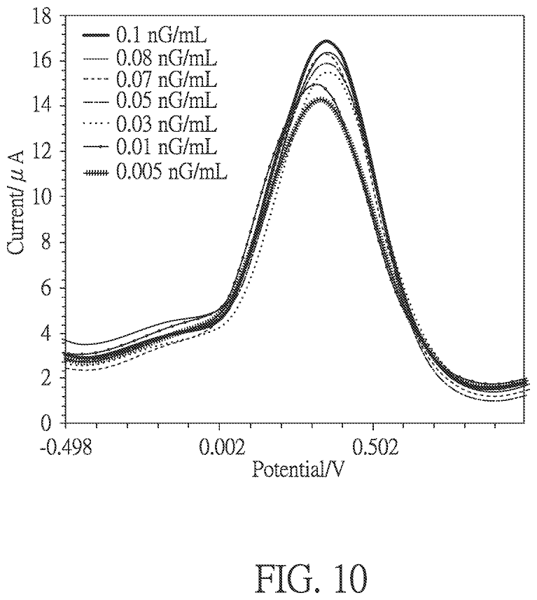

FIG. 10 shows an analytical performance of a comparative sensor using DPV. In the comparative sensor, SARS-CoV2 spike protein was immobilized on the graphene oxide layer on the graphene electrode to capture the anti-SARS-CoV2. The results shown in FIG. 10 corroborated that the observation of fall in peak current with the increasing concentration of protein is true, and concluded the specificity of the biosensor.

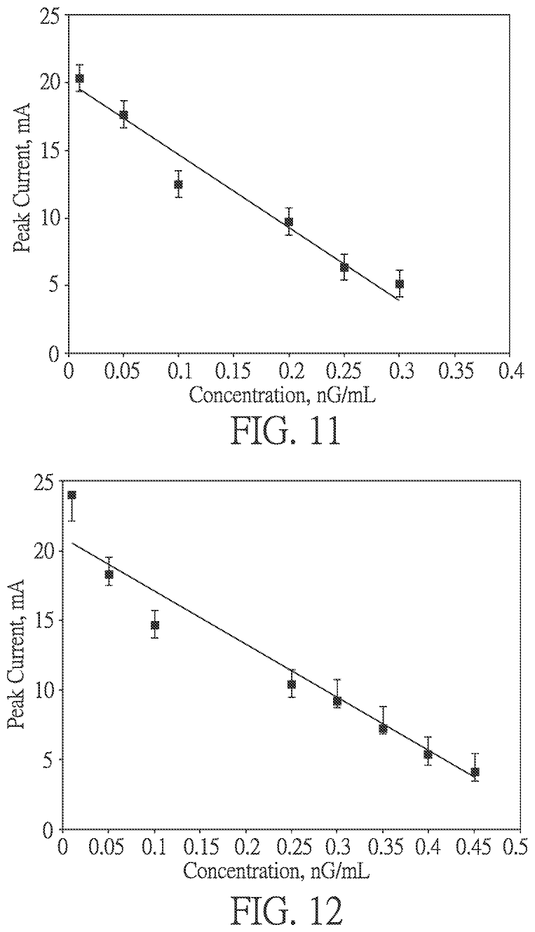

FIG. 11 and FIG. 12 respectively show the current vs. anti-SARS-CoV2 concentration curves of a biosensor prepared in Example 2 obtained by DPV based method and SWV based method. The results indicate that the LOD for DPV and SWV was estimated to be 10 fG mL⁻¹ and 14 fG mL⁻¹, respectively.

Herein, the antibody-based method of detection is employed to capture the viral spike protein; thus, the sensitivity and specificity of the fabricated biosensor is exceptional. Furthermore, periodical quantification of the spike protein is made possible by the present biosensor, which can provide an idea about the viral load in patients, thus leading to the monitoring and screening of effective drugs for covid-19.

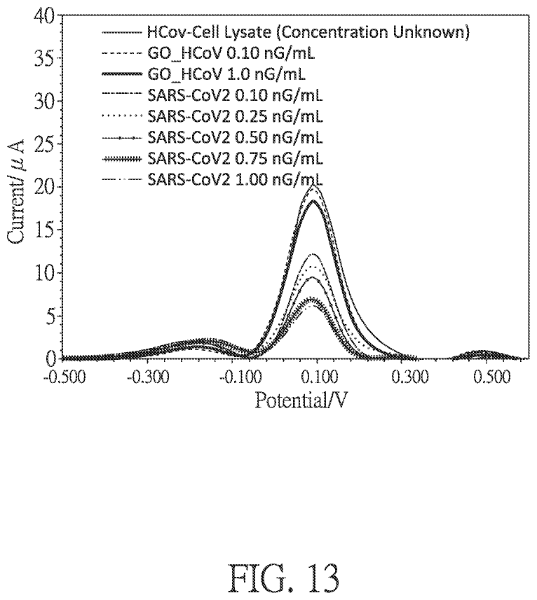

FIG. 13 shows an analytical performance of a biosensor prepared in Example 2 using differential pulse voltammetry (DPV), wherein Human coronavirus (HCoV 229E) and SARS CoV2 Spike Protein S1 were used as the analytes. In FIG. 13, HCov-Cell Lysate refers to the Human coronavirus (HCoV 229E) cell lysate, GO_HCoV refers to HCoV 229E antigen, and SARS-CoV2 refers to SARS CoV2 Spike Protein S1.

From the results shown in FIG. 13, high current was found in the group of HCoV 229E antigen and cell lysate, indicating that negligible binding of HCoV 229E antigen and cell lysate to the biosensor of the present disclosure. On the other hand, decreasing trend of current was found with the increasing concentration of protein in the group of SARS CoV2 Spike Protein S1, indicating that specificity of the biosensor of the present disclosure.

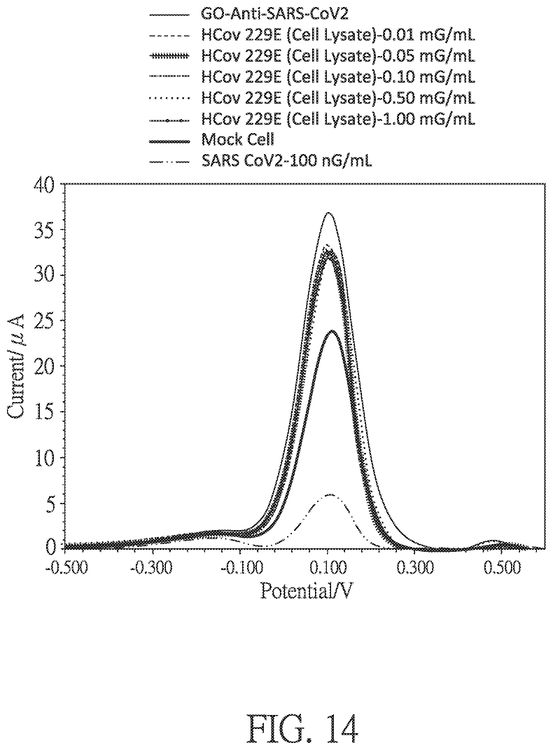

FIG. 14 shows an analytical performance of a biosensor prepared in Example 2 using differential pulse voltammetry (DPV), wherein Human coronavirus (HCoV 229E), Mock cell and SARS CoV2 Spike Protein S1 were used as the analytes. In FIG. 14, GO-Anti-SARS-CoV2 refers to the antibody of SARS-CoV2 as a negative control, HCoV 229E (Cell Lysate) refers to the Human coronavirus (HCoV 229E) cell lysate, and SARS-CoV2 refers to SARS CoV2 Spike Protein S1.

From the results shown in FIG. 14, high current was found in the group of HCoV 229E (Cell lysate), indicating that negligible binding of HCoV 229E cell lysate to the biosensor of the present disclosure. In the group of Mock cell, several non-specific markers were bound, but no significant change was found compared to SARS-CoV2 group. In the SARS-CoV2 group, decreasing trend of current was observed due to specific binding of the SARS CoV2 Spike Protein S1 to the biosensor of the present disclosure.

### Example 3

The biosensor of the present example is similar to that shown in Example 2, except for the following differences.

In the present example, the biosensor does not comprise the graphene oxide layer (i.e. the modification layer 122 on the working electrode 12 shown in FIG. 1). To prepare the graphene layer of the working electrode, graphene ink was modified with 1-pyrenebutyric acid N-hydroxysuccinimide ester (PBASE, linker used to introduce —COOH groups on graphene sheets), wherein PBASE is a derivative of succinic acid. Then, the EDC-NHS coupling as stated in Example 2 was performed to ensure maximum coverage of anti-SARS-CoV2 antibody. This method brings maximum accommodation of anti-SARS-CoV2 on working electrode due to the synergistic combination —COOH enriched graphene screen-printed layer with further EDC-NHS modification.

In conclusion, the biosensor of the present disclosure is prepared by a screen-printing method, which is simple to operate, cost-effective and avoids the usage of toxic photoresists which is employed in lithographic techniques. In addition, the biosensor of the present disclosure has the low LOD and/or the wide LR due to the covalent attachment between the bio-recognition elements and the graphene oxide of the modification layer. Thus, the biosensor of the present disclosure can detect and/or quantify the target biomolecules with remarkable sensitivity, specificity and/or rapid testing ability.

Although the present disclosure has been explained in relation to its embodiment, it is to be understood that many other possible modifications and variations can be made without departing from the spirit and scope of the disclosure as hereinafter claimed.

What is claimed is:

1. A biosensor, comprising:
a substrate;
a working electrode disposed on the substrate and comprising a graphene layer;
a counter electrode disposed on the substrate and adjacent to the working electrode; and
a bio-recognition layer disposed on the working electrode.

2. The biosensor of claim 1, wherein the working electrode further comprises a modification layer disposed on the graphene layer and comprising graphene oxide.

3. The biosensor of claim 1, wherein the counter electrode comprises graphene.

4. The biosensor of claim 1, wherein the working electrode is connected to the bio-recognition layer through a linker.

5. The biosensor of claim 4, wherein the linker is —CONH—.

6. The biosensor of claim 1, further comprising a reference electrode disposed on the substrate and electrically isolated from the working electrode.

7. The biosensor of claim 6, wherein the reference electrode is an Ag electrode or an Ag—AgCl composite electrode.

8. The biosensor of claim 1, wherein the bio-recognition layer comprises an antibody or lectin.

9. The biosensor of claim 8, wherein the lectin is Concanavalin A.

10. The biosensor of claim 8, wherein the antibody is an anti-severe acute respiratory syndrome coronavirus-2 (anti-SARS-CoV2) antibody.

11. A bio-sensing system, comprising:
a biosensor, comprising:
a substrate;
a working electrode disposed on the substrate and comprising a graphene layer;
a counter electrode disposed on the substrate and adjacent to the working electrode; and
a bio-recognition layer disposed on the working electrode; and
a detection device receiving signals from the biosensor.

12. The bio-sensing system of claim 11, wherein the detection device is an impedance analyzer or a voltammetry.

13. The bio-sensing system of claim 11, wherein the working electrode further comprises a modification layer disposed on the graphene layer and comprising graphene oxide.

14. The bio-sensing system of claim 11, wherein the counter electrode comprises graphene.

15. A method for preparing a biosensor, comprising the following steps:
providing a substrate;
forming a working electrode and a counter electrode on the substrate, wherein the counter electrode is adjacent to the working electrode, and the working electrode comprises a graphene layer; and
forming a bio-recognition layer on the working electrode.

16. The method of claim 15, wherein the working electrode or the counter electrode is prepared by a screen-printing process.

17. The method of claim 15, further comprising a step of forming a modification layer on the graphene layer before the step of forming the bio-recognition layer on the working electrode, wherein the modification layer comprises graphene oxide.

18. The method of claim 15, wherein the counter electrode comprises graphene.

19. The method of claim 15, wherein the working electrode is connected to the bio-recognition layer through a linker.

20. The method of claim 19, wherein the linker is —CONH—.
